(19) United States
(12) Patent Application Publication
(10) Pub. No.: US 2003/0236319 A1
(43) Pub. Date: Dec. 25, 2003

(54) Block copolymers for surgical articles

(76) Inventors: Hye-sung Yoon, Dae-jeon (KR); Tae-Hun Kim, Dae-jeon (KR); Myoung-Seok Koh, Dae-jeon (KR); Chong-taek Hong, Dae-jeon (KR); Chaul-min Pai, Dae-jeon (KR); Jung-nam Im, Dae-jeon (KR); Jang-il Seo, Dae-jeon (KR)

Correspondence Address: M. Wayne Western, THORPE, NORTH & WESTERN, L.L.P., P. O. Box 1219, Sandy, UT, 84091-1219, US

(21) Appl. No.: 10/307,139

(22) Filed: Nov. 27, 2002

(30) Foreign Application Priority Data
Jun. 25, 2002 (KR) 02-35834

(51) Int. Cl. C08F 20/00; A61F 13/00

(52) U.S. Cl. 523/122; 525/439

(57) ABSTRACT

The present invention relates to AB or ABA type block copolymers comprising p-dioxanone A blocks, and segmented copolymer B blocks comprising ε-caprolactone and trimethylene carbonate. The block copolymers are well suited for use as general medical and surgical articles requiring high flexibility, excellent knot-security and rapid absorption.

This application claims benefit of Korean Patent Application No. 02-35834 filed on Jun. 25, 2002.

### FIELD OF THE INVENTION

The present invention relates to block copolymers used for manufacture surgical articles, and more particularly, to block copolymers prepared by copolymerization of ε-caprolactone and trimethylene carbonate to produce segmented copolymers which are then reacted with p-dioxanone. The block copolymers of the present invention are suitable for making articles for general medical and surgical operations, i.e., sutures that require mechanical and biological properties.

### BACKGROUND OF THE INVENTION

Absorbable biocompatible polymers are widely used to manufacture surgical articles. Surgical articles, especially sutures, should have adequate tensile strength and biocompatibility. In addition, depending on their uses, sutures should be absorbable with appropriate absorption rates by the living body. Sutures should be also easy to handle during surgical operation.

Good handling properties of sutures are often closely related to their flexibility. In other words, a suture having a low flexibility cannot provide the desired knot security and is thus not suitable for application to minute body parts, such as suturing of blood vessels. Sutures used in operations on minute body parts such as blood vessels require good flexibility.

Sutures with appropriate knot-security are required for effective wound closure. In order to improve knot-security, multiple tying of knots has to be made with poor knot secured sutures, which causes harmful side effects such as tissue reaction and infection. However, knots made by sutures having high flexibility can remain secure and provide excellent knot-security without performing multiple-knot ties. In general, both good flexibility and knot-security are important physical properties of a suture. Furthermore, sutures having modifiable absorption rates, namely, the amount of time required for a suture absorbed in to the body by degradation, are often required so that sutures having a particular absorbability may be used selectively, depending on the application site of the body. For example, a 50% strength retention time may be 7 to 10 days, 14 days, 21 days, 28 days and etc. depending on the physicochemical properties of the suture materials.

The absorbable gut and collagen sutures have traditionally been made from natural materials, and they often produce unfavorable tissue reactions. Furthermore, it is difficult to obtain an expected strength retention time, because absorption rate of the natural materials is hard to predict.

Recently, it has been proposed to manufacture synthetic absorbable sutures containing greater than 60% polyglycolides. U.S. Pat. No. 5,431,679 describes block copolymers comprising polyglycolide, trimethylene carbonate and caprolactone. Sutures made from these block copolymers show poor flexibility.

U.S. Pat. No. 5,854,383 discloses segmented copolymers of aliphatic polyesters based on lactone monomers, glycolide, trimethylene carbonate and ε-caprolactone. Although these segmented copolymers provide for improved flexibility compared to those of U.S. Pat. No. 5,431,679, they show poor knot-security.

U.S. Pat. No. 4,052,988 discloses the preparation of a p-dioxanone homopolymer and its use as an absorbable surgical suture. This synthetic suture exhibits outstanding mechanical and biological properties that make it a viable candidate to replace natural sutures, such as surgical gut and collagen, for numerous applications. Furthermore, U.S. Pat. No. 5,047,048 discloses the preparation of copolymers of p-dioxanone and ε-caprolactone that provide improved flexibility compared to the p-dioxanone homopolymer. However, it has not reached the desired degrees of flexibility.

As described above, there have been many efforts for improving characteristic of sutures. However, above patents and research do not achieve to improve suture materials having flexibility, knot security and controlling of absorption properties, which are important requirements of a suture. Therefore, the present invention provides a block copolymer with excellent knot security, flexibility and appropriate absorption properties, which helps overcome the disadvantages of currently commercialized surgical materials.

### SUMMARY OF THE INVENTION

The present invention relates to block copolymers prepared by reacting segmented copolymers made from ε-caprolactone and trimethylene carbonate with p-dioxanone monomers. The block copolymers of the present invention are well suited for uses as surgical articles requiring high flexibility, excellent knot-security and rapid absorption. The present invention also relates to methods for preparing such block copolymers.

### BRIEF DESCRIPTION OF THE DRAWING

FIG. 1 illustrates a synthesis scheme for preparing the block copolymers of the present invention.

### DETAILED DESCRIPTION OF THE INVENTION

The present invention relates to block copolymers comprising p-dioxanone blocks and segmented copolymer blocks prepared from ε-caprolactone and trimethylene carbonate.

The present invention also relates to a method for producing a block copolymer comprising the steps of: 1) polymerizing ε-caprolactone and trimethylene carbonate to produce a segmented copolymer; 2) adding p-dioxanone monomers to said segmented copolymer, and 3) co-polymerizing said segmented copolymer with the p-dioxanone monomers to form a block copolymer.

The present invention is described in detail hereunder.

One embodiment of the present invention is a block copolymer comprising a polydioxanone block and a segmented copolymer block comprising ε-caprolactone copolymerized with trimethylene carbonate. In the present invention, two types of blocks having different structures are combined advantageously to form a block copolymer.

The block copolymers of the present invention can be prepared as diblock or triblock copolymers. Diblock copolymers can be prepared by polymerizing polydioxanone with a segmented copolymer made from ε-caprolactone and trimethylene carbonate. Triblock copolymers can be prepared by using polydioxanone as the end blocks and the segmented copolymer made from ε-caprolactone and trimethylene carbonate as the center blocks.

The block copolymers of the present invention comprise a p-dioxanone unit as expressed by the following formula (1) and a segmented copolymer unit made of ε-caprolactone and trimethylene carbonate as expressed by the following formula (2),

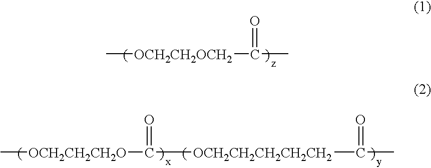

Wherein x, y, and z respectively represents an integer between 10 to 10⁴.

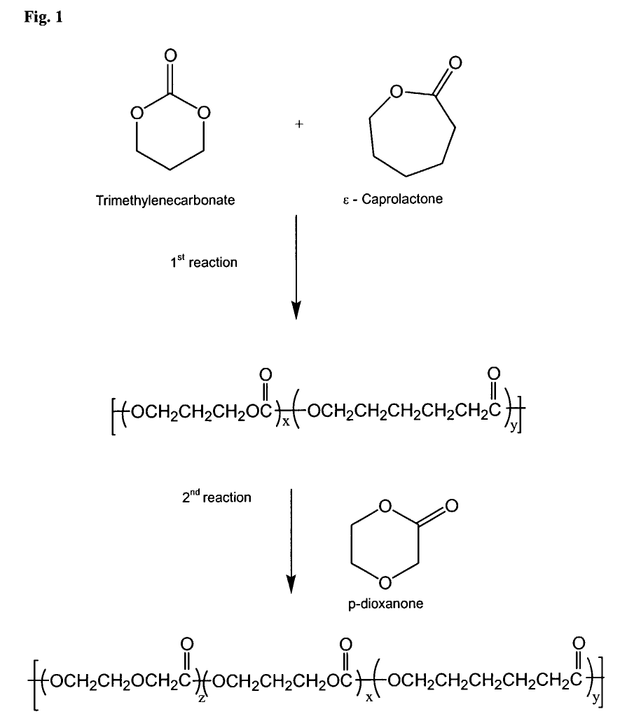

FIG. 1 represents a synthesis scheme of preparing the block copolymers of the present invention.

For example, one process for preparing the block copolymers of the present invention comprises two steps. First, copolymerization of ε-caprolactone and trimethylene carbonate is carried at a temperature of from 120 to 180° C., preferably 150 to 170° C., to form a segmented copolymer. Second, the resulting segmented copolymer is then reacted with p-dioxanone monomers to produce the block copolymers of the present invention, as shown in FIG. 1. In other words, the segmented copolymer (formula (2)) comprising ε-caprolactone and trimethylene carbonate obtained from the first copolymerization step is then reacted with p-dioxanone monomers to produce a block copolymer comprising a p-dioxanone unit of formula (1) and a segmented copolymer unit of formula (2),

In a preferred embodiment of the present invention, the first copolymerization to produce the segmented copolymer of formula (2) is carried out at a temperature of from 120 to 180° C., preferably from 150 to 170° C., for 0.5 to 2 hours. In the second copolymerization, p-dioxanone is then added to the segmented copolymer at a temperature of from 130 to 160° C. and reacted for 1 to 3 hours. The reaction temperature is preferably lowered to 90° C. and the reaction is preferably performed for 5 to 20 hours. Furthermore, the reaction temperature may be lowered to 80° C. and the reaction performed for 3 to 5 days, preferably for 3 to 4 days.

In the second copolymerization step to produce the block copolymer, the reaction is preferably performed via multiple steps because p-dioxanone decomposes at high temperature. Therefore, after the segmented copolymer is formed, the second reaction is preferably performed at a lower temperature.

The block copolymers of the present invention are synthesized in the presence of an organometal catalyst and an initiator, which are typically used in a ring opening polymerization. The organometal catalyst is preferably tin 2-ethylhexanoate, and is preferably present in the monomer mixture at a molar ratio of monomer to catalyst ranging from 10,000:1 to 100,000:1. The initiator used in the present invention typically is an alkanol, such as diols and polyols, a glycol, a hydroxyacid, or an amine, and is preferably present in the monomer mixture at a molar ratio of monomer to initiator ranging from about 500:1 to about 5,000:1. The polymerization reaction is carried out at an appropriate temperature and for an appropriate amount of time until the desired molecular weight and viscosity are achieved.

Depending on the initiator used in the synthesis, the block copolymers formed in the present invention can be diblock or triblock copolymers. Diblock copolymers of the present invention can be prepared by polymerizing polydioxanone with segmented copolymers made from ε-caprolactone and trimethylene carbonate. Triblock copolymers of the present invention have polydioxanone as the end blocks and the segment copolymers made from ε-caprolactone and trimethylene carbonate as the center blocks. The initiator used for producing triblock copolymers of the present invention is a bifunctional initiator, such as diols having hydroxyl groups at both sides and p-dioxanone at both ends. For example, when ε-caprolactone and trimethylene carbonate are polymerized in the presence of diethylene glycol (hereafter referred to as “DEG”) as an initiator, both hydroxyl groups of the DEG react with each of ε-caprolactone and trimethylene carbonate to form a segmented copolymer which may further react with p-dioxanone. p-dioxanone is then added and reacted at the both ends of the segmented copolymer to produce a triblock copolymer.

Preferably, the molar ratio of ε-caprolactone/trimethylene carbonate in the segmented copolymer as shown in above formula (2) is within the range of 5:95 to 95:5, and more preferably within the range of 90:10 to 10:90. A higher molar ratio of trimethylene carbonate in the segmented copolymer correlates to a higher flexibility, while a higher molar ratio of ε-caprolactone in the segmented copolymer correlates to a longer period of strength retention. Therefore, the molar ratio of ε-caprolactone/trimethylene carbonate can be controlled to provide varying degrees of flexibility, strength retention or absorption rate. The segmented copolymers of the present invention preferably have an inherent viscosity within the range of 0.5 dL/g to 2.1 dL/g, more preferably from 0.7 dL/g to 1.8 dL/g as measured in a 0.1 g/dL solution of hexafluoroisopropanol (HFIP) at 25° C.

Preferably, the content of the p-dioxanone blocks as shown in above formula (1) in the block copolymer of the present invention is within the range of 5 to 95 mole %, more preferably from 60 to 90 mole %. An amount below 5 mole % fails to obtain adequate strength and absorption rate to the extent that the copolymer may no longer be suitable for use as a surgical suture due to a crystallinity of lower than 10%.

The copolymer of the present invention can be random or block copolymers but the block copolymer is much more preferred. The crystallinity of a random copolymer is too low to have a knot tensile strength of greater than 10,000 psi, which is required as a surgical suture.

The block copolymers of the present invention have an inherent viscosity ranging from 0.8 dL/g to 4.0 dL/g, preferably from 1.0 dL/g to 3.5 dL/g as measured in a 0.1 g/dL solution of hexafluoroisopropanol (HFIP) at 25° C. The block copolymer of the present invention has a Young's modulus below 180,000 psi, preferably below 140,000 psi.

Since the block copolymers of the present invention are prepared by polymerization of the segmented copolymer with p-dioxanone, the total crystalline domains are reduced compared to p-dioxanone homopolymers which thereby provides for low degrees of crystallinity. In other words, due to the increased non-crystalline domains of block copolymers of the present invention have a lower Young's modulus compared to the Young's modulus of a p-dioxanone homopolymer. The low Young's modulus of the block copolymers correlates with a higher flexibility, more stable knot-security. Furthermore, the block copolymers have higher strength due to the physical crosslinks between the non-crystalline domains. In addition, a reduction in the degree of crystallinity also correlates with faster absorption. Therefore, the block copolymers of the present invention have a low Young's modulus, high flexibility and faster absorption rates while monitoring good strength.

The block copolymers of the present invention are useful for the preparation of implantable medical and surgical articles, especially absorbable surgical sutures, surgical screws, surgical staples, surgical clips, pins, anastomosis rings, surgical meshes, prosthetic devices and the like. The block copolymers of the present invention can be injection-molded into surgical screws, surgical staples, surgical clips, pins, and anastomosis rings. The block copolymers of the present invention can be melt-extruded and drawn to produce filaments such as sutures. A surgical mesh can be prepared by knitting and/or weaving or non-weaving the filaments.

The block copolymers of the present invention are suitable for making surgical articles such as prosthetic devices, and especially surgical sutures that require a low Young's modulus and high knot-security. Additionally, the sutures made from the block copolymers of the present invention can be attached to needles. Among possible suture applications of the block copolymers of the present invention, monofilament sutures are preferred because they provide less tissue drag and have lower infection rate.

Monofilament sutures prepared from the block copolymers of the present invention have straight tensile strengths of at least 45,000 psi, preferably from 45,000 to 70,000 psi, and more preferably from 49,000 to 53,000 psi. The knot-pull strength of monofilament sutures prepared from the block copolymers of the present invention is at least 25,000 psi, preferably from 30,000 to 50,000 psi, and more preferably from 30,000 to 33,000 psi. The Young's modulus of monofilament sutures prepared from the block copolymers of the present invention are less than 180,000 psi, and are preferably from 30,000 to 180,000 psi. Sutures prepared from the block copolymers of the present invention have a 50% strength retention time of 7 to 10 days, thus being suitable in situations requiring rapid absorption.

As described above, the block copolymers of the present invention prepared by reacting p-dioxanone monomers with segmented copolymers made from ε-caprolactone and trimethylene carbonate are suitable for use as sutures and have excellent properties such as a low Young's modulus, high flexibility, high knot strength and high knot-security. The block copolymers of the present invention are also useful for other materials requiring biocompatibility and rapid absorption.

The block copolymers obtained by the present invention have excellent strength, flexibility and absorbability and are suitable to be used as medical articles such as surgical sutures, surgical screws, surgical staples, surgical clips, pins, anastomosis rings, surgical meshes, prosthetic devices and the like. The block copolymers obtained by the present invention are especially useful as monofilament synthetic absorbable sutures. Furthermore, the block copolymers of the present invention have high strength, flexibility, excellent knot-security, and a 50% strength retention period from 7 to 10 days and so are useful for surgical articles which demand rapid absorption.

The following Examples and Comparative Examples are intended to further illustrate the present invention without limiting its scope.

The monomers and block copolymers used in the Examples and Comparative Examples were analyzed by the following methods: NMR (Bruker AVANCE DPX 400) and GC (HP5890) for chemical structure and purity thereof; a viscometer (Cannon) for inherent viscosity as measured in a 0.1 g/dL solution of hexafluoroisopropanol (HFIP) at 25° C.; x-ray diffraction (Rigaku Tenki RAD/B) for degree of crystallinity. Strength change was measured after storing in phosphate buffer solution (pH=7.27) at 37° C. over time.

The block copolymer was molten, spun, drawn and annealed to prepare sutures using conventional extrusion techniques. Methods for measuring the physical properties of sutures-knot security

Knot security was measured in terms of the knot slippage ratio. A surgeon's knot (2=1=1) was selected for the knot tying method. The knotted sutures were placed on a tensile strength tester and pulled apart until knot breakage occurred or the knot slipped. After ten measurements, the ratio of the number of knots slipped to the total number of the knots tied indicates the knot slippage ratio. Thus, the less the ratio, the better the knot security of the suture.

Methods for Measuring the Physical Properties of Sutures—Flexibility

A flexibility data of sutures are based on Young's moduli derived from measuring linear tensile strength

TABLE 1

| Physical properties | Method |
|---|---|
| Straight tensile | ASTM D2256 |
| strength | Used machine: Instron |
| | Length of sample: 130 mm |
| | Measuring Rate: 130 mm/min |
| Knot-pull strength | EP(European Pharmacopeias) |
| | (Monofilament suture) |
| | Used machine: Instron |
| | Length of sample: 100 mm |
| | Measuring Rate: 100 mm/min |
| Diameter, mm | EP regulation, Diameter |
| Young's modulus | Used machine: Instron |
| | Stress-strain curve |
| Knot-security | Used machine: Instron |
| | Type of knots: Surgeon's knot (2 = 1 = 1) |
| | Length of sample: 5 mm |
| | Measuring Rate: 500 mm/min |
| | Standard for determination: knot slippage ratio = |
| | (sliding knotted samples no./ |
| | 10 knotted samples) 100 |

### EXAMPLE 1

Preparation of Diblock Copolymer Comprising ε-caprolactone/trimethylene carbonate at 10/90 by mole and 90 mole % of p-dioxanone

To a dried flask was added 138.6 g of trimethylene carbonate and 15.4 g of ε-caprolactone. The contents of the reaction flask, fitted with a mechanical stirrer, were held and dried under vacuum. After drying, to the reaction mixture was added 0.8 mL of tin 2-ethylhexanoate (0.63 M solution in toluene) and 1.8 g of lauryl alcohol by syringe. The reaction mixture was reacted in an oil bath while maintaining a temperature of 160° C. for 2 hours under a nitrogen atmosphere. The reactor was purged with nitrogen and 1346.4 g of p-dioxanone was added while stirring. The temperature of reaction mixture was lowered to 150° C. and maintained at this temperature for 1 hour, then lowered to 90° C. and maintained at this temperature for 16 hours, then further lowered to 80° C. and maintained at this temperature for 72 hours. The block copolymer was isolated and dried under vacuum to remove any unreacted monomer.

The composition obtained is polydioxanone (PDO)/polytrimethylene carbonate (PTMC)/polycaprolactone (PCL), (89/9.0/1.1), as determined by ¹H-NMR. The degree of crystallinity of the copolymer was 23%, as determined by X-ray diffraction. The inherent viscosity of the diblock copolymer was 2.3 dL/g as measured in a 0.1 g/dL solution of hexafluoroisopropanol (HFIP) at 25° C. The properties of the block copolymer are summarized in Table 2.

### EXAMPLE 2

Preparation of Diblock Copolymer Comprising ε-caprolactone/trimethylene carbonate at 25/75 by mole and 90 mole % of p-dioxanone

To a dried flask was added 112.2 g of trimethylene carbonate and 41.8 g of ε-caprolactone. The contents of the reaction flask, fitted with a mechanical stirrer, were held and dried under vacuum. After drying, to the reaction mixture was added 0.8 mL of tin 2-ethylhexanoate (0.63 M solution in toluene) and 1.8 g of lauryl alcohol by syringe. The reaction mixture was reacted in an oil bath while maintaining a temperature of 160° C. for 2 hours under a nitrogen atmosphere. The reactor was purged with nitrogen and 1346.4 g of p-dioxanone was added while stirring. The temperature of reaction mixture was lowered to 150° C. and maintained at this temperature for 1 hour, then lowered to 90° C. and maintained at this temperature for 16 hours, then further lowered to 80° C. and maintained at this temperature for 72 hours. The block copolymer was isolated and dried under vacuum to remove any unreacted monomer.

The composition obtained is polydioxanone (PDO)/polytrimethylene carbonate (PTMC)/polycaprolactone (PCL), (89/8.5/2.5), as determined by ¹H-NMR. The degree of crystallinity of the copolymer was 25%, as determined by X-ray diffraction. The inherent viscosity of the diblock copolymer was 2.9 dL/g as measured in a 0.1 g/dL solution of hexafluoroisopropanol (HFIP) at 25° C. The properties of the block copolymer are summarized in Table 2.

### EXAMPLE 3

Preparation of Diblock Copolymer Comprising ε-caprolactone/trimethylene carbonate at 75/25 by mole and 90 mole % of p-dioxanone

To dried flask was added with 37.4 g of trimethylene carbonate and 125.5 g of ε-caprolactone. The contents of the reaction flask, fitted with a mechanical stirrer, were dried under vacuum. After drying, to the reaction mixture was added 1 mL of tin 2-ethylhexanoate (0.63 M solution in toluene) and 2 g of lauryl alcohol via syringe. The reaction mixture was reacted in an oil bath while maintaining a temperature of 160° C. for 2 hours under a nitrogen atmosphere. The reactor was purged with nitrogen and 1346.4 g of p-dioxanone was added while stirring. The temperature of reaction mixture was lowered to 150° C. and maintained at this temperature for 1 hour, then lowered to 90° C. and maintained at this temperature for 16 hours, then lowered to 80° C. and maintained at this temperature for 72 hours. The block copolymer was isolated and dried under vacuum to remove any unreacted monomer.

The composition of the polydioxanone (PDO)/polytrimethylene carbonate (PTMC)/polycaprolactone (PCL) obtained was 89/3.5/7.5 as determined by ¹H-NMR. The degree of crystallinity of the copolymer was 29%, which was determined by X-ray diffraction. The inherent viscosity of the diblock copolymer was 2.3 dL/g as measured in a 0.1 g/dL solution of hexafluoroisopropanol (HFIP) at 25° C. The properties of the block copolymer are summarized in Table 2.

### EXAMPLE 4

Preparation of Diblock Copolymer Comprising ε-caprolactone/trimethylene carbonate at 50/50 by mole and 80 mole % of p-dioxanone

To a dried flask was added with 204 g of trimethylene carbonate and 228 g of ε-caprolactone. The contents of the reaction flask, fitted with a mechanical stirrer, were dried under vacuum. After drying, to the reaction mixture was added 2.29 mL of tin 2-ethylhexanoate (0.63 M solution in toluene) and 4 g of lauryl alcohol via syringe. The reaction mixture was reacted in an oil bath while maintaining a temperature of 160° C. for 2 hours under a nitrogen atmosphere. The reactor was purged with nitrogen and 1632 g of p-dioxanone was added while stirring. Further reactions were performed as in Example 1. The properties of the block copolymer are summarized in Table 2.

### EXAMPLE 5

Preparation of Diblock Copolymer Comprising ε-caprolactone/trimethylene carbonate at 25/75 by mole and 80 mole % of p-dioxanone

To a dried flask was added with 306 g of trimethylene carbonate and 114.1 g of ε-caprolactone. The contents of the reaction flask, fitted with a mechanical stirrer, were dried under vacuum. After drying, to the reaction mixture was added 2.29 mL of tin 2-ethylhexanoate (0.63 M solution in toluene) and 4 g of lauryl alcohol via syringe. The reaction mixture was reacted in an oil bath while maintaining a temperature of 160° C. for 2 hours under nitrogen atmosphere. The reactor was purged with nitrogen and 1632 g of p-dioxanone was added while stirring. Further reactions were performed as in Example 1. The properties of the block copolymer are summarized in Table 2.

### EXAMPLE 6

Preparation of Diblock Copolymer Comprising ε-caprolactone/trimethylene carbonate at 75/25 by mole and 80 mole % of p-dioxanone

To a dried flask were added 102 g of trimethylene carbonate and 342.3 g of ε-caprolactone. The contents of the reaction flask, fitted with a mechanical stirrer, were dried under vacuum. After drying, to the reaction mixture was added 2.29 mL of tin 2-ethylhexanoate (0.63 M solution in toluene) and 4 g of lauryl alcohol via syringe. The reaction mixture was reacted in an oil bath while maintaining a temperature of 160° C. for 2 hours under nitrogen atmosphere. The reactor was purged with nitrogen and 1632 g of p-dioxanone was added while stirring. Further reactions were performed as in Example 1. The properties of the block copolymer are summarized in Table 2.

### EXAMPLE 7

Preparation of Triblock Copolymer Comprising p-dioxanone end Blocks and a Segmented Copolymer Made from Trimethylene Carbonate and ε-caprolactone as the Center Block, wherein the Composite Ratio is 45/(7/3)/45 by mole

To a dried flask were added 112.2 g of trimethylene carbonate and 41.5 g of ε-caprolactone. The contents of the reaction flask, fitted with a mechanical stirrer, were dried under vacuum. After drying, to the reaction mixture was added 1.8 mL of tin 2-ethylhexanoate (0.63 M solution in toluene) and 1.8 g of diethylene glycol (DEG) via syringe. The reaction mixture was reacted in an oil bath while maintaining a temperature of 170° C. for 1 hour. The reactor was purged with nitrogen and the temperature of the reaction mixture was lowered to 160° C., followed by addition of 1346.4 g of p-dioxanone while stirring. The reaction temperature was maintained at 160° C. for 1 hour under nitrogen atmosphere. The temperature of reaction mixture was lowered to 90° C. and maintained at this temperature for 16 hours, and then further lowered to 80° C. and maintained at this temperature for 72 hours. The block copolymer was isolated and dried under vacuum to remove any unreacted monomer.

The composition of the polydioxanone (PDO)/polytrimethylene carbonate (PTMC): polycaprolactone (PCL): polydioxanone (PDO) obtained was 44:8.0/3.0/45 as determined by ¹H-NMR. The inherent viscosity of the triblock copolymer was measured in a 0.1 g/dL solution of hexafluoroisopropanol (HFIP) at 25° C. The properties of the block copolymer are summarized in Table 2.

TABLE 2

| | Composition | Inherent Viscosity | Crystallinity |
|---|---|---|---|
| Polymer | (PDO/PTMC/PCL) | (dL/g) | (%) |
| Example 1 | 89/9.0/1.1 | 2.4 | 23 |
| Example 2 | 89/8.5/2.5 | 2.9 | 25 |
| Example 3 | 89/3.5/7.5 | 2.3 | 29 |
| Example 4 | 79.2/10.4/10.4 | 1.8 | 20 |
| Example 5 | 79/15.6/5.4 | 2.3 | 23 |
| Example 6 | 79.1/5.0/15.9 | 2.0 | 26 |
| Example 7* | 44/(8/3)/45 | 2.0 | 26 |
| Control** | 100 | 2.5 | 32 |

*Example 6: Triblock copolymer comprising PDO/(PTMC/PCL)/PDO
**Control: PDO homopolymer

As shown in Table 2, both diblock copolymers and triblock copolymers of the present invention have comparative inherent viscosities in the range of from 1.8 to 2.9, and crystallinities of 20 to 29% which is about 3 to 12% lower than that of PDO homopolymers. When the amount of PDO is the same, an increase in the amount of PTMC correlates with a reduced crystallinity and an increase in the amount of PCL correlates with an increased crystallinity.

### Comparative Example 1

Preparation of Random Copolymer Comprising ε-caprolactone, Trimethylene Carbonate and p-dioxanone

To a dried flask was added 306 g of trimethylene carbonate, 114.1 g of ε-caprolactone, and 612 g of p-dioxanone. The contents of the reaction flask, fitted with a mechanical stirrer, were dried under vacuum. After drying, to the reaction mixture was added 0.8 mL of tin 2-ethylhexanoate (0.63 M solution in toluene) and 1.8 g of lauryl alcohol via syringe. The reaction mixture was reacted in an oil bath while maintaining a temperature of 160° C. for 2 hours. The reactor was purged with nitrogen and the temperature of reaction mixture was lowered to 150° C. and maintained at this temperature for 1 hour under nitrogen atmosphere, then further lowered to 90° C. and maintained at this temperature for 16 hours. The block copolymer was isolated and dried under vacuum to remove any unreacted monomer.

The composition of the polydioxanone (PDO)/polytrimethylene carbonate (PTMC)/polycaprolactone (PCL) obtained was 62/38/10 as determined by ¹H-NMR. The degree of crystallinity of the copolymer was 15% which was determined by X-ray diffraction. The inherent viscosity of the random copolymer was 1.7 dL/g. The crystallinity of the random copolymer was too low to be used as a suture due to low knot strength of 10,000 psi.

### Comparative Example 2

Preparation of Copolymer Comprising p-dioxanone/ε-caprolactone at 90/10 by Mole

To a dried 500 mL flask was added 41.0 g of ε-caprolactone. The contents of the reaction flask, fitted with a mechanic stirrer, were dried under vacuum. After drying, to the reaction mixture was added 0.34 mL of tin 2-ethylhexanoate (0.63 M solution in toluene) and 2 g of lauryl alcohol via syringe. The reaction mixture was reacted in an oil bath while maintaining a temperature of 160° C. for 1 hour. The reactor was purged with nitrogen and the temperature of reaction mixture was lowered to 110° C. and then 265.2 g of p-dioxanone was added while stirring. The reaction temperature was maintained at 110° C. for 4 hours, lowered to 90° C. and maintained at this temperature for 24 hours, then further lowered to 80° C. and maintained at this temperature for 72 hours. The block copolymer was isolated and dried under vacuum to remove any unreacted monomer.

### EXAMPLE 8

Preparation of Monofilament Using Block Copolymers

The block copolymers from Examples 1, 2, 3, 5, 6 and 7 were extruded into monofilaments under the following conditions; see Table 3, to compare copolymer properties. The copolymer properties are summarized in Table 4.

TABLE 3

| | | |
|---|---|---|
| Category | Unit | Condition |

Melt Extrusion

| | | |
|---|---|---|
| Extruder screw | rpm | 6 |
| Pump | rpm | 11 |
| Barrel temperature, 1 zone | ° C. | 175 |
| Barrel temperature, 2 zone | ° C. | 177 |
| Barrel temperature, 3 zone | ° C. | 179 |
| Pump temperature | ° C. | 179 |
| Pump melt temperature | ° C. | 180 |
| Spinneret melt temperature | ° C. | 180 |
| Barrel Pressure | Kgf/cm² | 70 |
| Spinneret Pressure | Kgf/cm² | 60 |
| Pump size | cc/rev | 2.0 |
| Quench bath temperature | ° C. | 22 |

Stretching & Orientation

| | | |
|---|---|---|
| Draw oven temperature (1^{st}/2^{nd}/3^{rd}) | ° C. | 90/95/95 |
| First godet | mpm | 5.4 |
| Second godet | mpm | 26.4 |
| Third godet | mpm | 27.9 |
| Winding speed | rpm | 25 |
| Draw ratio | | 4.63 |

TABLE 4

| Block copolymer | Example 1 | | Example 2 | | Example 3 | | Example 5 | | Example 6 | | Example 7 | |
|---|---|---|---|---|---|---|---|---|---|---|---|---|
| Composition | 89/9.0/1.1 | | 89/8.5/2.5 | | 89/3.5/7.5 | | 79/15.6/5.4 | | 79.1/5/15.9 | | 44/8/3/45 | |
| (PDO/PTMC/PCL) | | | | | | | | | | | | |
| Diameter | 0.546 | mm | 0.548 | mm | 0.534 | mm | 0.550 | mm | 0.530 | mm | 0.549 | mm |
| Knot pull strength | 35,000 | psi | 32,000 | psi | 30,000 | psi | 30,000 | psi | 31,000 | psi | 33,000 | psi |
| Straight tensile | 58,000 | psi | 53,000 | psi | 50,000 | psi | 49,000 | psi | 51,000 | psi | 53,000 | psi |
| strength | | | | | | | | | | | | |
| Young's Modulus | 100,000 | psi | 120,000 | psi | 150,000 | psi | 90,000 | psi | 110,000 | psi | 130,000 | psi |

| | | | | | | |
|---|---|---|---|---|---|---|
| Elongation | 67% | 64% | 70% | 68% | 72% | 66% |

As shown in the above Table 4, the block copolymer of the present invention has a straight tensile strength of greater than 49,000 psi, a high knot strength of at least 30,000, a Young's Modulus of below 150,000 psi, and excellent flexibility. Also when the amount of PDO was same, a higher molar ratio of trimethylene carbonate in the segmented copolymer correlates to a higher flexibility.

### EXAMPLE 9

Property Comparison of the Copolymers of the Present Invention, Copolymer of Comparative Example 2, and Commercial Monofilament Suture

The copolymers from Examples 1 (PDO/PTMC/PCL) and 4 (PDO/PTMC/PCL), and the copolymer from Comparative Example 2 (PDO-PCL) were extruded into a monofilament under the conditions listed in Table 3 to compare copolymer properties such as Young's Modulus and knot-security. The commercial monofilament used was MONOCRYL™, made from glycolide and ε-caprolactone.

TABLE 5

| | | | Comparative | MONO- |
|---|---|---|---|---|
| Monofilament | Example 1 | Example 4 | Example 2 | CRYL ™ |
| Young's | 100,000 | 90,000 | 200,000 | 230,000 |
| Modulus(psi) | | | | |
| Knot-security(%) | 30 | 50 | 100 | 80 |

As shown in Table 5, the block copolymers of the present invention exhibit not only a significant reduction in Young's Modulus as compared to MONOCRYL™ but also higher flexibility. Furthermore, the block copolymers of the present invention show better knot-security (3-5 out of 10 knots failed) than MONOCRYL™ (8 knots failed).

### EXAMPLE 10

Comparison in Breaking Strength Retention of the Block Copolymers of the Present Invention, Copolymer of Comparative Example 2, and Commercial Monofilament

The change of strength retention in vitro was determined after storage in phosphate buffer, pH 7.27 at 37° C. by comparison with the initial strength. The results are summarized in Table 6.

TABLE 6
Breaking strength retention depending on a storing period

| | | | Comparative | |
|---|---|---|---|---|
| Monofilament | Example 1 | Example 3 | Example 2 | MONOCRYL ™ |
| Initial | 100 | 100 | 100 | 100 |
| After 7 days | 60 | 77 | 90 | 70 |
| After 10 days | 49 | 55 | 90 | 60 |
| After 14 days | 30 | 40 | 80 | 55 |

As shown in Table 6, the 50% strength retention period of the block copolymers of the present invention was 10 days, which was much faster than that of the copolymer of Comparative Example 2 or MONOCRYL™. Furthermore, an increase in the amount of PTMC, when the amount of PDO was same, correlates to a reduction in the 50% strength retention period as shown in Examples 1 and 3.

The above description will enable one skilled in the art to make the block copolymers of the present invention by reacting p-dioxanone monomers with segmented copolymers made from ε-caprolactone and trimethylene carbonate and to thereby make sutures having excellent properties such as low Young's modulus, high flexibility, high knot strength and high knot-security. Although they are described to show the functionality of block copolymers of the present invention, these descriptions are not intended to be exhaustive. It will be immediately apparent to one skilled in the art that various modifications may be made without departing from the scope of the invention, which is limited only by the following claims and their functional equivalents.

We claim:

1. An AB type di-block copolymer comprising a p-dioxanone A block, and a segmented copolymer B block comprising ε-caprolactone and trimethylene carbonate.

2. The diblock copolymer of claim 1, wherein the content of said p-dioxanone A blocks are within the range of 5 to 95 mole %.

3. The diblock copolymer of claim 2, wherein the content of said p-dioxanone blocks are within the range of 60 to 90 mole %.

4. The diblock copolymer of claim 1, wherein the content of said segmented copolymer B block made from ε-caprolactone and trimethylene carbonate is within the range of 5 to 95 mole %.

5. The diblock copolymer of claim 1, wherein the molar ratio of ε-caprolactone/trimethylene carbonate in said segmented copolymer B block is within the range of 5/95 to 95/5.

6. The diblock copolymer of claim 5, wherein the molar ratio of ε-caprolactone/trimethylene carbonate in said segmented copolymer B block is within the range of 10/90 to 90/10.

7. The diblock copolymer of claim 1 has an inherent viscosity within the range of 0.8 to 4.0 dL/g as measured in a 0.1 g/dL solution of hexafluoroisopropanol at 25° C.

8. The diblock copolymer of claim 7 has an inherent viscosity within the range of 1.0 to 3.5 dL/g as measured in a 0.1 g/dL solution of hexafluoroisopropanol at 25° C.

9. The diblock copolymer of claim 1 has a Young's modulus of at least 180,000 psi.

10. An AB type di-block copolymer comprising a p-dioxanone A block, and a segmented copolymer B block comprising ε-caprolactone and trimethylene carbonate, wherein the content of said p-dioxanone A blocks are within the range of 5 to 95 mole %, and the molar ratio of ε-caprolactone/trimethylene carbonate in said segmented copolymer B block is within the range of 5/95 to 95/5.

11. A surgical article comprising an AB type di-block copolymer comprising a p-dioxanone A block, and a segmented copolymer B block comprising ε-caprolactone and trimethylene carbonate.

12. The surgical article of claim 11, wherein the surgical article is a member selected from the group consisting of surgical sutures, surgical screws, surgical staples, surgical clips, pins, anastomosis rings, surgical meshes and prosthetic devices.

13. The surgical article of claim 12, wherein said surgical suture is attached to needles.

14. The surgical article of claim 12, wherein the straight tensile strength of said surgical suture is within the range of from 45,000 to 70,000 psi.

15. The surgical article of claim 12, wherein the knot-pull strength of said surgical suture is within the range of from 30,000 to 50,000 psi.

16. The surgical article of claim 12, wherein the Young's modulus of said surgical suture is in the range of from 30,000 to 180,000 psi.

17. A surgical article comprising an AB type di-block copolymer comprising a p-dioxanone A block, and a segmented copolymer B block comprising ε-caprolactone and trimethylene carbonate, wherein the content of said p-dioxanone A blocks are within the range of 5 to 95 mole %, and the molar ratio of ε-caprolactone/trimethylene carbonate in said segmented copolymer B block is within the range of 5/95 to 95/5.

18. The surgical article of claim 17, wherein the surgical article is a member selected from the group consisting of surgical sutures, surgical screws, surgical staples, surgical clips, pins, anastomosis rings, surgical meshes and prosthetic devices.

19. The surgical article of claim 18 has a straight tensile strength within the range of from 45,000 to 70,000 psi, a knot-pull strength within the range of from 30,000 to 50,000 psi and a Young's modulus within the range of from 30,000 to 180,000 psi.

20. An ABA type tri-block copolymer comprising a p-dioxanone A block, and a segmented copolymer B block comprising ε-caprolactone and trimethylene carbonate.

21. The tri-block copolymer of claim 20, wherein the content of said p-dioxanone A blocks are within the range of 10 to 90 mole %.

22. The triblock copolymer of claim 20, wherein the content of said segmented copolymer B block made from ε-caprolactone and trimethylene carbonate is within the range of 10 to 90 mole %.

23. The triblock copolymer of claim 20, wherein the molar ratio of ε-caprolactone/trimethylene carbonate in said segmented copolymer B block is within the range of 5/95 to 95/5.

24. The triblock copolymer of claim 23 wherein the molar ratio of ε-caprolactone/trimethylene carbonate in said segmented copolymer B block is within the range of 10/90 to 90/10.

25. The triblock copolymer of claim 20, wherein the inherent viscosity of said block copolymer is in the range of from 0.8 to 4.0 dL/g, as measured in a 0.1 g/dL solution of hexafluoroisopropanol at 25° C.

26. The triblock copolymer of claim 20 has an inherent viscosity within the range of from 1.0 to 3.5 dL/g, as measured in a 0.1 g/dL solution of hexafluoroisopropanol at 25° C.

27. The triblock copolymer of claim 20 has a Young's modulus of at least 180,000 psi.

28. An ABA type tri-block copolymer comprising a p-dioxanone A block, and a segmented copolymer B block comprising ε-caprolactone and trimethylene carbonate, wherein the content of said p-dioxanone A blocks are within the range of 10 to 90 mole %, and the molar ratio of ε-caprolactone/trimethylene carbonate in said segmented copolymer B block is within the range of 5/95 to 95/5.

29. A surgical article comprising an ABA type tri-block copolymer comprising a p-dioxanone A block, and a segmented copolymer B block comprising ε-caprolactone and trimethylene carbonate.

30. The surgical article of claim 29, wherein the surgical article is a member selected from the group consisting of surgical sutures, surgical screws, surgical staples, surgical clips, pins, anastomosis rings, surgical meshes and prosthetic devices.

31. The surgical article of claim 30, wherein said surgical suture is attached to needles.

32. The surgical article of claim 30, wherein the straight tensile strength of said surgical suture is within the range of from 45,000 to 70,000 psi.

33. The surgical article of claim 30, wherein the knot-pull strength of said surgical suture is within the range of from 30,000 to 50,000 psi.

34. The surgical article of claim 30, wherein the Young's modulus of said surgical suture is in the range of from 30,000 to 180,000 psi.

35. A surgical article comprising an ABA type tri-block copolymer comprising a p-dioxanone A block, and a segmented copolymer B block comprising ε-caprolactone and trimethylene carbonate, wherein the content of said p-dioxanone A blocks are within the range of 10 to 90 mole %, and the molar ratio of ε-caprolactone/trimethylene carbonate in said segmented copolymer B block is within the range of 5/95 to 95/5.

36. The surgical article of claim 35, wherein the surgical article is a member selected from the group consisting of surgical sutures, surgical screws, surgical staples, surgical clips, pins, anastomosis rings, surgical meshes and prosthetic devices.

37. The surgical article of claim 35 has a straight tensile strength within the range of from 45,000 to 70,000 psi, a knot-pull strength within the range of from 30,000 to 50,000 psi and a Young's modulus within the range of from 30,000 to 180,000 psi.

38. A process for preparing a block copolymer comprising the steps of:
(a) polymerizing ε-caprolactone and trimethylene carbonate to produce a segmented copolymer block; and
produce a block copolymer comprising p-dioxanone blocks and segmented copolymer blocks comprising ε-caprolactone and trimethylene carbonate.

39. An AB or ABA type block copolymer comprising p-dioxanone A blocks, and segmented copolymer B blocks comprising ε-caprolactone and trimethylene carbonate prepared from the process of claim 38.

40. A block copolymer comprising an A block of p-dioxanone units represented by formula (1) and a B block comprising a segmented copolymer of ε-caprolactone and trimethylene carbonate, said B block is represented by formula (2), 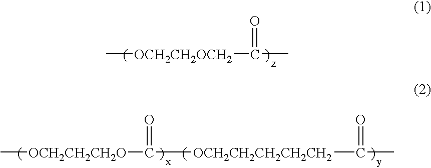
Wherein x, y, and z respectively represents an integer between 10 to 10⁴

41. The block copolymer of claim 38 is an AB type di-block copolymer containing 5 to 95 mole % of said A blocks and 5 to 95 mole % of said B blocks.

42. The block copolymer of claim 38 is an ABA type tri-block copolymer containing 10 to 90 mole % of said A blocks and 10 to 90 mole % of said B blocks.
